(19) United States
(12) Patent Application Publication
(10) Pub. No.: US 2010/0069722 A1
(43) Pub. Date: Mar. 18, 2010

(54) INTUBATION LARYNGOSCOPE WITH DETACHABLE BLADES

(76) Inventors: Michael Shalman, Ashdod (IL); Leonid Lukov, Beer-Sheva (IL)

Correspondence Address: NIXON & VANDERHYE, PC, 901 NORTH GLEBE ROAD, 11TH FLOOR, ARLINGTON, VA, 22203, US

(21) Appl. No.: 12/310,133

(22) PCT Filed: Aug. 15, 2007
(86) PCT No.: PCT/IB2007/002357

§ 371 Date: Nov. 17, 2009

(30) Foreign Application Priority Data
Aug. 15, 2006 (US) 11/504062

(51) Int. Cl. A61B 1/267

(52) U.S. Cl. 600/193; 600/194

(57) ABSTRACT

An intubation laryngoscope having reusable and disposable blades adapted for both right and left-handed operation, an illumination system mounted on the laryngoscope handle, and an improved fastening appliance for detachable connection of the blades to the laryngoscope handle.

### BACKGROUND

1. Field of the Invention

The present invention relates to the intubation laryngoscopes having reusable and disposable blades adapted for right- and left-handed operation, an illumination system mounted on a laryngoscope handle, and improved fastening appliance for detachable connection of the blades to the laryngoscope handle.

2. Prior Art

The most widely spread design of intubation laryngoscope 100, for example fabricated by Welch Allyn company (see enclosed FIGS. 1 to 5), comprises a blade set, including four detachable blades of curvilinear Macintosh profile of various size designed for operations with children and adults. Each blade 101 has blade distal end portion 102 designed to expose a tracheal entrance in order to insert an endotracheal tube therein, blade proximal end portion 103 designed for detachable connection with laryngoscope handle 104 through blade holder 105, upper longitudinal part 106 to be interacted with patient's tongue, and lateral longitudinal part 107 disposed laterally and below relative to the upper longitudinal part. The position of lateral longitudinal part 107, on the left or on the right side relative to upper longitudinal part 106, determines what operator's hand, right or left, respectively, is used for inserting the endotracheal tube, while another operator's hand holds handle 104. All shown prior art versions (FIGS. 1, 2, 4, 5) are designed for the right-handed operation because lateral longitudinal part 107 is disposed on the left side of upper longitudinal part 106. Blade longitudinal parts 106, 107 have the form of a thinwalled sheet of various outlines and curvature. Blade proximal end portion 103 is provided with fastening appliance 108 affixed to the blade, designed for detachable connecting blade 101 to blade holder 105 and made in the form of a substantially thickened member comprising hook element 109, two springy ball stoppers 110 and 111, and a seat, wherein proximal end 112 of fiberoptic light guide 113 is held. Light guide 113 is extended distally of fastening appliance 108, passes through window 114 in blade lateral longitudinal part 107, and its distal face 115 emitting an illumination light is located beneath upper longitudinal part 106 near blade distal end portion 102. In the embodiment of company Heine, named Greenline-profile (FIGS. 4, 5), the fiberoptic light guide is housed in closed sheath 116. Blade holder 105 includes socket 117 designed for placing blade fastening appliance 108 therein and bar 118 to be engaged with hook element 108. The illumination system of laryngoscope comprises an illumination lamp, a lamp cartridge including light switch 119, batteries hosed inside handle 104 and designed for feeding the lamp, as well as mentioned fiberoptic light guide. The design of the noted lamp cartridge is disclosed in U.S. Pat. No. 6,277,068 B1. Switch 119 is movable relative to the lamp cartridge and turns on the lamp, when fastening appliance 108 is completely connected with blade holder 105. To turn off the lamp, handle 104 and blade 101 should be folded by two operator's hands.

The disadvantage of the above intubation laryngoscope is its relatively high cost caused with the considerable complexity of the illumination system and the fastening means for detachable connecting the blade to the blade holder because each of the four blades of the blade set is equipped with the fastening appliance and fiberoptic light guide. The blade complexity also is caused with the peculiarity of the fastening appliance and the means for affixing the light guide to the blade. The high cost of illumination system is caused also with the complex design of the lamp cartridge including two springs, mutually movable components and sealing means. Another disadvantage is hindered conditions for washing the blade because of the presence of hard accessible numerous gaps and pockets between the blade walls and the light guide in Welsh Allyn version shown in FIGS. 1, 2. In Greenline version (FIGS. 4, 5), this disadvantage is mainly eliminated due to using sheath 116. However the sheath introduction substantially heightens the blade cost. Another disadvantage is the blade incapability of both right-handed and left-handed operating. Typically, the blades are fabricated for the right-handed operation, which is very inconvenient for left-handed anesthesiologists. Another disadvantage is high requirements to the blade strength caused with the peculiarity of blade loading and its fastening in the blade holder resulting in heightened stresses in blade material at the zone of the blade fastening to the blade holder. These stresses hinder the use of inexpensive plastic disposable blades, whose strength is not sufficient and increase the weight of the conventional metal blades.

Patent Application US 2005/0159649 A1 discloses a laryngoscope including a handle and a blade integrally formed of a single piece of material. The light source unit is deprived of a light guide and attached to the blade opposite to the handle. The absence of the light guide simplifies the laryngoscope design. However, the arrangement of the light source beneath the handle predetermines the considerable distance between the light source and the blade distal end portion. This demands the considerable increase of the light power and is permissible mainly for the blades having substantially straightened profile, whose application, as distinct from above Macintosh profile, is restricted because of the hindered operation with patient's tongue. Another disadvantage is shading the tracheal entrance by the endotracheal tube during the intubation procedure worsening the tracheal entrance vision. Another disadvantage is the location of the light source on the blade requiring mounting the light source on each blade. The integrally fabricated handle and blade allow excluding a blade holder and a blade fastening appliance, thereby simplifying design. However, in the disposable version, this substantially heightens the cost of a disposable component because it includes also the handle, and, in reusable version, where each blade of the blade set must be fabricated with its own handle, that also substantially heightens the laryngoscope set cost. Another disadvantage is incapability of using the laryngoscope both for right-handed and for left-handed operations.

Patent Application US 2002/0082478 A1 discloses a laryngoscope including a receiving body with a receiving portion, wherein an elongated blade is gripped by a cam means and a set of jaws. This receiving portion fulfils a part of a blade holder. A handle is pivotably connected to the receiving portion and laterally spaced apart from it. The position of the blade in the receiving portion is adjustable. The illumination means including a light guide is located in the blade. The disadvantage of the laryngoscope is substantial complexity of the means for connecting the blade to the handle because of the presence of the bulky receiving portion equipped with the cam means and the set of jaws, and complicated design of blade part inserted into the receiving portion. Another disadvantage is the substantial complicated blade design, which includes the illumination means and, in version embodiment, also viewing means. This considerably impedes the use of the blade as a disposable component. The laryngoscope can not be used both for right-handed and for left-handed operations. The lateral arrangement of the handle with respect to the blade creates a transverse torque causing an additional inconvenience for anesthesiologist.

### SUMMARY OF THE INVENTION

The objective of the present invention is providing the capability of both right-handed and left-handed operation of the laryngoscope.

Another objective is simplifying the design and reducing the cost of the laryngoscope illumination means.

Another objective is protecting the illumination means as a whole from infecting during the intubation procedure.

Another objective is simplifying the design and reducing the cost of the laryngoscope fastening means for detachable connection of the blade to the handle.

Another objective is considerable simplifying the design and reducing the cost of the laryngoscope blades.

Another objective is easing the procedure of the blade washing and disinfection.

Another objective is substantial reducing the requirements for the blade strength and corresponding reducing the material expenditure for the blade fabrication and the blade weight.

Another objective is providing a universal laryngoscope capable of operating both with reusable metal blades and with disposable plastic blades.

The above noted objectives are accomplished with an intubation laryngoscope comprising a handle and at least one detachable blade. The latter has a blade distal end portion designed to expose a tracheal entrance in order to insert an endotracheal tube therein, a blade proximal end portion to be detachable connected to the handle by a fastening means, an upper longitudinal part to be interacted with patient's tongue, and a lateral longitudinal part disposed laterally and below relative to the upper longitudinal part. The left side or right side position of the lateral longitudinal part determines what operator's hand, right or left, respectively, is used for inserting the endotracheal tube, while another operator's hand holds the handle. Each of the blade end portions is adapted for using as the blade distal end portion for immediate exposing the tracheal entrance, and there is a capability of previous turning the blade through 180° to thereby convert the blade proximal end portion into the blade distal end portion and simultaneously to change the lateral disposition of the lateral longitudinal part relative to the upper longitudinal part in order to change the operator's hand to be used for inserting the endotracheal tube. Therewith, the fastening means, including a blade holder, disposed at a distal end of the handle, is capable of detachable connecting the blade to the handle regardless of what blade end portion is chosen as the blade distal end portion. Also a laryngoscope illumination means is capable of illuminating the zone of the endotracheal tube insertion regardless of what blade end portion is chosen as the blade distal end portion. Thus the capability of both right-handed and left-handed operation of the laryngoscope, depending on operator's choice, is achieved.

The blade longitudinal parts have the form of a thinwalled sheet of various outlines and curvature, the fastening means includes a blade fastened section in the form of a thinwalled sheet portion, and at least one blade engagement element immovable relative to the blade, disposed at the blade fastened section and made as a recess in the blade fastened section. Therewith, the blade fastened section and the blade engagement element are made of the thinwalled sheet of blade longitudinal parts and located in the limits of the blade proximal end portion. Moreover the blade holder includes a holder slit adapted to inserting the thinwalled sheet blade fastened section therein with an insignificant gap to partial fix the blade in the blade holder. Besides, the blade holder includes at least one holder engagement element adapted to detachable engagement with the blade engagement element, movable relative to the blade holder and capable of entering into engagement with the blade engagement element to fix the blade fastened section in the holder slit and to thereby attach the blade to the blade holder and handle. The holder engagement element also is capable of coming out from the engagement with the blade engagement element to decouple the blade fastened section and the holder and to thereby detach the blade from the blade holder and handle. As a result, as distinct from the prior art, the fastening means components, located on the blade, consist only of the thinwalled sheet blade fastened section, which, in version embodiment, presents the segment of the blade longitudinal part, and the recesses thereon. Therewith the fixing movable engagement element is disposed only on the blade holder allowing at least fourfold decrease in the number of the fixing movable engagement elements in comparison with the prior art (see the laryngoscopes of Welsh Allyn and Heine companies), where these elements are located on each of the four blades of the laryngoscope set. Thus, the substantial simplifying the design and reducing the cost of the laryngoscope fastening means is achieved.

The blade engagement element is disposed on each of the blade end portions so that each of the blade end portions also is adapted for using as the blade proximal end portion. Therewith, the design of the blade engagement element, made as a recess in the blade fastened section, allows unobstructed introducing the blade into patient's mouth, the normal function of the blade end portions as a means for the exposure of tracheal entrance, and unobstructed inserting the endotracheal tube into trachea. As a result, each of the blade end portions is capable of applying both as the blade distal end portion designed for immediate exposing the tracheal entrance and as the blade proximal end portion designed for connecting to the handle through the blade holder.

In version embodiment, the blade is symmetric relative to a mid transverse plane of the blade.

In another version embodiment, the blade fastened section presents a segment of the blade upper longitudinal part in the limits of each of the blade end portions, the blade engagement element is made as an engaging opening, and the holder engagement element is made as a movable engaging projection entering the engaging opening to attach the blade to the blade holder. The mentioned movable engaging projection presents a ball of a springy ball stopper, which enters the engaging opening under the action of the stopper spring and comes out from the engaging opening overcoming the spring resistance as a result of trailing the blade by operator.

The movable engaging projection also can be embodied as an element located on a movable control member mounted in the blade holder, controlled by operator and provided with a fixing means to fix it, when the blade is attached to the blade holder. Therewith, the movable control member is made as a lever rotatable around a lever axle mounted in the blade holder.

In version embodiment, the blade fastened section is straight and flat and the holder slit is located in a single plane.

The mutual arrangement of the components of the described fastening means provides for inserting the blade fastened section into the holder slit, thereafter the movable engaging projection enters the engaging opening thereby fixing the blade fastened section in the holder slit and attaching the blade to the holder and handle.

In version embodiment, the external surface of the blade fastened section is used as the blade engagement element, the holder engagement element presents a clamping member, which, in the blade attached state, presses the external surface of blade fastened section against opposite wall of the holder slit thereby fixing the blade in the holder by means of the friction force arising between the external surface of blade fastened section and the holder slit wall.

In version embodiment, the fastening means includes two blade engagement elements disposed at one blade fastened section and correspondently two holder engagement elements movable relative to the blade holder.

In version embodiment, the offered laryngoscope comprises a set of the detachable blades of different length including blades for children and adults, and each of the detachable blades is provided with the mentioned blade fastened sections and the blade engagement elements.

In version embodiment, the holder slit is capable of receiving the thinwalled sheet blade fastened sections of both a reusable metal blade, whose sheet thickness amounts in the order of about 1.5 mm, and a disposable plastic blade, whose sheet thickness amounts in the order of about 3 mm, therewith the holder slit size is designed for receiving the fastened section of plastic blade, while the fastened section of metal blade comprises at least one local stamped convexity providing the total thickness of the metal blade fastened section equal to the thickness of plastic blade fastened section. Thus, the certain universality of the offered laryngoscope is achieved.

According to the present invention, the illumination means is constantly connected with the handle and along with the handle detachable from the blades, allowing unobstructed previous turning the blade through 180° and providing the needed illumination of the zone of endotracheal tube insertion for any of the blade end portions chosen for using as the distal end portion. The absence of any connection with the illumination means, along with the above simplification of the fastening means elements located on the blade, allow considerable simplifying the blade design and reducing by several times the blade cost in the comparison with the prior art. Moreover, this allows excluding the hard accessible gaps and pockets in the blade design thereby easing and cheapening the procedure of the blade washing and disinfection.

The offered illumination means include a single light extender mounted in the blade holder, extended from the blade holder in a distal direction and located below the blade upper longitudinal part in the proximity to the blade upper longitudinal part and the blade lateral longitudinal part. Therewith, the single light extender is adapted for operation with any of the several detachable blades of the blade set including the detachable blades of various length designed for children and adults. Such illumination means, in comparison with the Prior Art (see Patent Application US 2005/0159649 A1), does not demand so considerable increase of the light power, is compatible with the curvilinear blades of Macintosh type and is not shaded with the endotracheal tube.

In version embodiment, the offered light extender has a strengthened sheath located beneath the blade upper longitudinal part adjacent to it, providing a reliable mechanical protection of the light extender and allowing using the strengthened sheath as a support of the blade resulting in substantial decrease of the requirements for blade strength. This enables substantial decreasing the thickness of blade walls and blade weight, as well as providing the favorable conditions for using inexpensive and easy processed material of blades as plastic. Eventually, this leads to additional considerable reducing the blade cost resulting in throughout extension of the use of disposable plastic blades due to the drastic reduction of the blade cost and heightening the plastic blade reliability due to prevention of the blade breakdowns.

The strengthened sheath is covered with a transparent plastic protector preventing the light extender and strengthened sheath from infecting in patient's mouth and used as a disposable part.

In version embodiment, the light extender and its strengthened sheath are made as an integral detachable unit allowing its detachment from the blade holder to sterilize after infecting in patient's mouth.

In version embodiment, the illumination means includes one transmitting light guide mounted on the blade, disposed along the blade adjacent to the upper longitudinal part and lateral longitudinal part, having a transmitting light guide distal end located proximally of the blade end distal portion, as well as a transmitting light guide proximal end located distally of the blade end proximal portion in front of a distal light emitting surface of the blade holder in the immediate vicinity of it, providing the light transmitting from the holder light emitting surface through the transmitting light guide to the zone of endotracheal tube insertion, therewith, turning said blade through 180° leads to converting the transmitting light guide distal end into the transmitting light guide proximal end providing the same light transmitting from the holder light emitting surface to the zone of endotracheal tube insertion.

In version embodiment, there are two transmitting light guides mounted on each blade, disposed along said blade adjacent to said upper longitudinal part and lateral longitudinal part, and each of these two transmitting light guides is designed for operation with one certain blade end portion providing the light transmitting from a holder distal light emitting surface to said zone of endotracheal tube insertion, therewith, turning said blade through 180° leads to putting into operation another transmitting light guide providing the same light transmitting from said holder distal light emitting surface to said zone of endotracheal tube insertion.

In version embodiment, the blade upper longitudinal part consists of four variously inclined straight flat segments forming a blade profile approximately similar to Macintosh profile, including two relatively short peripheral segments, distal and proximal, adapted for immediate exposing the tracheal entrance and for using them as the blade fastened section, as well as two internal segments adapted for interacting with patient's tongue, for favorable arrangement of the light extender thereunder and for passing the illumination light from the light extender distal face to the zone of endotracheal tube insertion located beneath the peripheral distal segment, therewith the straightness of the peripheral segments is needed for their easy insertion into the holder slit and the straightness of the internal segments is needed for passing the illumination light from the light extender distal face to the zone of endotracheal tube insertion. As applying to the longest blade the light extender distal face is located near a transient zone between two internal segments.

The offered fastening means does not allow to use the light switch, which is used in the prior art, specifically in the laryngoscopes of Welsh Allyn and Heine companies, allowing turning on the illumination lamp by the blade. In this connection, the present illumination means comprises the new switch system including the lamp cartridge located movably beneath the feeding batteries. The lamp cartridge includes a cartridge housing having an inner thread in its lower portion for screwing in the illumination lamp. A cartridge contactor is housed within the cartridge housing and has an upper end to be constantly contacted with the lower electrical contact of the batteries and a lower end to be constantly contacted with the illumination lamp. The cartridge contactor is insulated from the cartridge housing by cartridge insulators housed inside the cartridge housing. A light switch is located at the upper end of the laryngoscope handle and allows operator to turn on and turn off the illumination lamp. The light witch comprises: a switch housing located above the batteries, made as a handle cap and connected to the handle upper end by means of thread; a switch insulator housed inside the switch housing, protruding downward from the switch housing and to be contacted with the upper electric contact of the batteries, when illumination lamp is turned off; a switch contactor rotatably mounted on the switch housing by means of thread, having the constant electric contact with the illumination lamp through the switch housing, the metal handle and the cartridge housing, and comprising a contacting protrusion capable of axial moving within the switch insulator as a result of rotating the switch contactor. The light switch is provided with the limiter of the switch contactor rotation mounted on the switch housing and determining the angular position “On” and “Off” of the switch contactor. A lower spring is disposed between the lamp cartridge and the blade holder and designed to provide the reliable constant electric contact between the cartridge contactor upper end and the batteries lower electrical contact, as well as between the batteries upper electric contact and the contacting protrusion of the switch contactor, when the illumination lamp is turned on. Besides, the lower spring serves as a lower support of the batteries and the lamp cartridge providing their reliable mounting in laryngoscope. Such use of the lower spring is novelty. Thus, the present light switch has no any contact with the laryngoscope blades allowing the unobstructed turning on and turning off the illumination light independently of the blade position. As distinct from the Prior Art, the present light switch may be controlled by the same operator's hand, which holds the handle. The present lamp cartridge along with the lamp switch comprise substantially less part number than the lamp cartridge assembly of Prior Art and these parts demand less fabrication accuracy. As a result, the noted components of the present laryngoscope are cheaper. As distinct from the Prior Art, the present lamp cartridge has no mutually movable parts and is fully sealed thereby preventing the parts from jamming and from getting a liquid inward during the lamp cartridge disinfecting. It should be noted, the offered light switch system is necessary for the functioning the offered laryngoscope, but it can be also used for another laryngoscopes providing mainly the same advantages.

### BRIEF DESCRIPTION OF THE DRAWINGS

FIGS. 1 to 5 show the intubation laryngoscopes and their components of Prior Art, wherein:

FIGS. 1 to 3 show the intubation laryngoscope and its components of company “Welsh Allyn”.

FIGS. 4, 5 show the intubation laryngoscope and its blade of company “Heine”.

The drawings presented in the following relate to the present invention.

FIG. 6 shows the cross-sectional view of the blade holder with the shortest plastic blade and the shortened anti-infective plastic protector.

FIG. 7 shows the cross-sectional view of the blade holder with the longest metal blade and the lengthened anti-infective plastic protector.

FIG. 8 shows the cross-sectional view of the intubation laryngoscope with the longest plastic blade and lengthened anti-infective plastic protector.

FIG. 9 shows the close-up cross-sectional view of the blade holder.

FIG. 10 shows the close-up cross-sectional view of the intubation laryngoscope without the blade.

FIGS. 11 to 13 show the disposable plastic blade applicable both for the right-handed and for left-handed operations.

FIGS. 14 to 16 show the laryngoscope assembly in 3D graphic presentation, wherein:

FIG. 14 show the laryngoscope assembly with the plastic blade ready for the right-handed insertion of the endotracheal tube into patient's trachea.

FIG. 15 shows the laryngoscope plastic blade applicable both for the right-handed and for the left-handed operations.

FIG. 16 show the laryngoscope assembly with the plastic blade ready for the left-handed insertion of the endotracheal tube into patient's trachea.

FIG. 17 shows the laryngoscope assembly with the metal blade ready for the right-handed insertion of the endotracheal tube into patient's trachea.

FIG. 18 shows the assembly of the lamp cartridge of the laryngoscope illumination means.

FIG. 19 shows the subassembly of the control lever of the laryngoscope fastening means designed for detachable connecting the blade to the blade holder.

FIG. 20 shows the springy ball stopper used in the laryngoscope fastening means.

FIG. 21 shows the anti-infective lengthened disposable plastic protector of the laryngoscope illumination means.

FIG. 22 shows the metal blade applicable both for the right-handed and for the left-handed operations.

FIGS. 23, 24 show the cross-sectional views of the anti-infective shortened disposable plastic protector of the laryngoscope illumination means.

FIGS. 25, 26 show the cross-sectional views of the anti-infective lengthened disposable plastic protector of the laryngoscope illumination means.

FIGS. 27 to 30 show the version of the intubation laryngoscope and its components including the metal reusable blade applicable both for the right-handed and left-handed operations and provided with the transmitting light guide, wherein:

FIG. 27 shows the assembly of the intubation laryngoscope ready for the left-handed insertion of the endotracheal tube into patient's trachea.

FIG. 28 shows the assembly of the intubation laryngoscope ready for the right-handed insertion of the endotracheal tube into patient's trachea.

FIG. 29 shows the subassembly of the blade holder.

FIG. 30 shows the metal reusable blade applicable both for the right-handed and left-handed operations and provided with the transmitting light guide.

FIG. 31 shows the intubation laryngoscope subassembly adapted to the connection with the disposable blade comprising the anti-infective protection of the laryngoscope illumination means.

FIG. 32 shows the disposable plastic blade comprising the anti-infective protector of the laryngoscope illumination means made in the form of the proximal blade socket.

FIGS. 33 to 36 show the intubation laryngoscope and its components providing for the use of the anti-infective protection socket of the disposable blade also as the means for fastening the blade to the blade holder, wherein:

FIG. 33 shows the laryngoscope assembly ready for the operation.

FIG. 34 shows the blade holder subassembly with the distal protrusion designed for inserting into the blade proximal socket.

FIG. 35 shows the disposable blade with the proximal socket to be connected with the holder distal protrusion and serving as an anti-infective protector.

FIG. 36 shows the intersected disposable blade.

FIGS. 37 to 39 show the intubation laryngoscope and its components providing for connecting the proximal socket of the reusable blade to the blade holder, wherein:

FIG. 37 shows the laryngoscope assembly ready for the operation.

FIG. 38 shows the reusable blade with the proximal socket adapted to its washing.

FIG. 39 shows the blade holder with the restricted distal protrusion.

FIGS. 40 to 43 show the intubation laryngoscope wherein the existing blade is combined with the offered fastening means using the springy ball stopper as a fixing means, wherein:

FIG. 40 shows the subassembly including the handle and the blade holder.

FIG. 41 shows the blade holder.

FIG. 42 shows the existing blade of company “Heine” with changed fastening means adapted to interacting with the offered blade holder.

FIG. 43 shows the assembly of the intubation laryngoscope ready for the operation.

FIGS. 44 to 47 show the intubation laryngoscope wherein the existing blade is combined with the offered fastening means using the latch as a fixing means, wherein:

FIG. 44 shows the subassembly including the handle and the blade holder.

FIG. 45 shows the blade holder.

FIG. 46 shows the existing blade of company “Heine” with changed fastening means adapted to interacting with the offered blade holder and including the blade engagement element movable relative to the blade.

FIG. 47 shows the assembly of the intubation laryngoscope ready for the operation.

FIGS. 48 to 51 show an intubation laryngoscope version, wherein the strengthening sheath of a single light extender is made as an integral part of the blade holder and the engagement appliance includes a fastening nut.

### DETAILED DESCRIPTION OF THE INVENTION

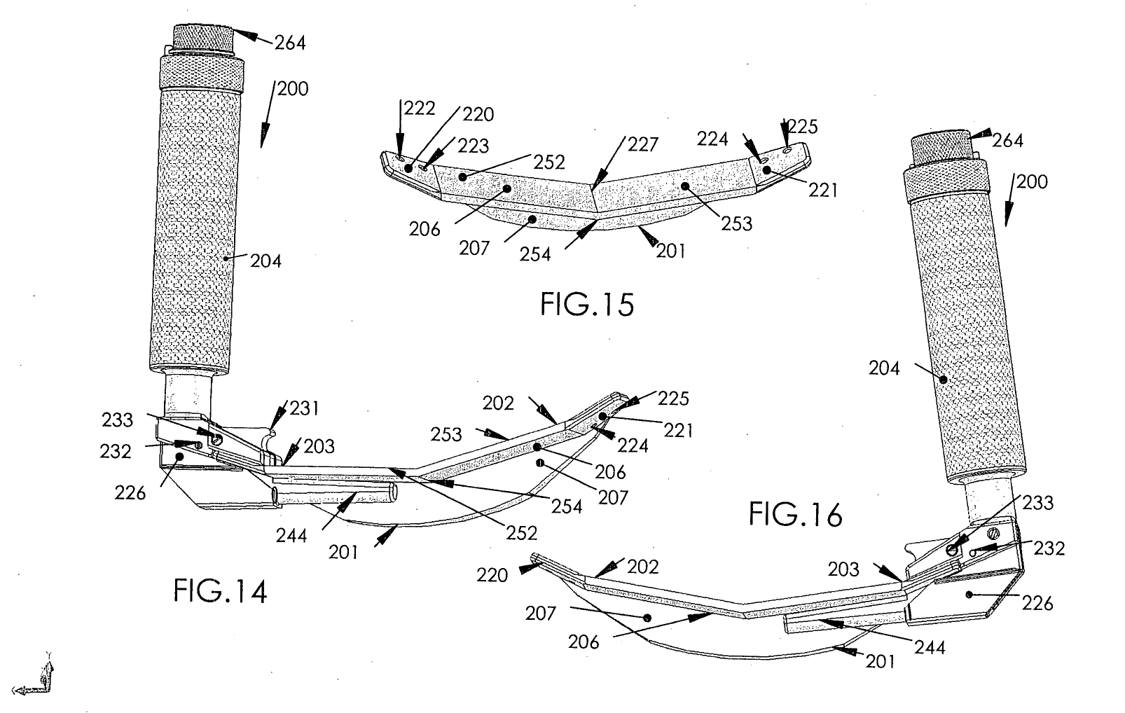

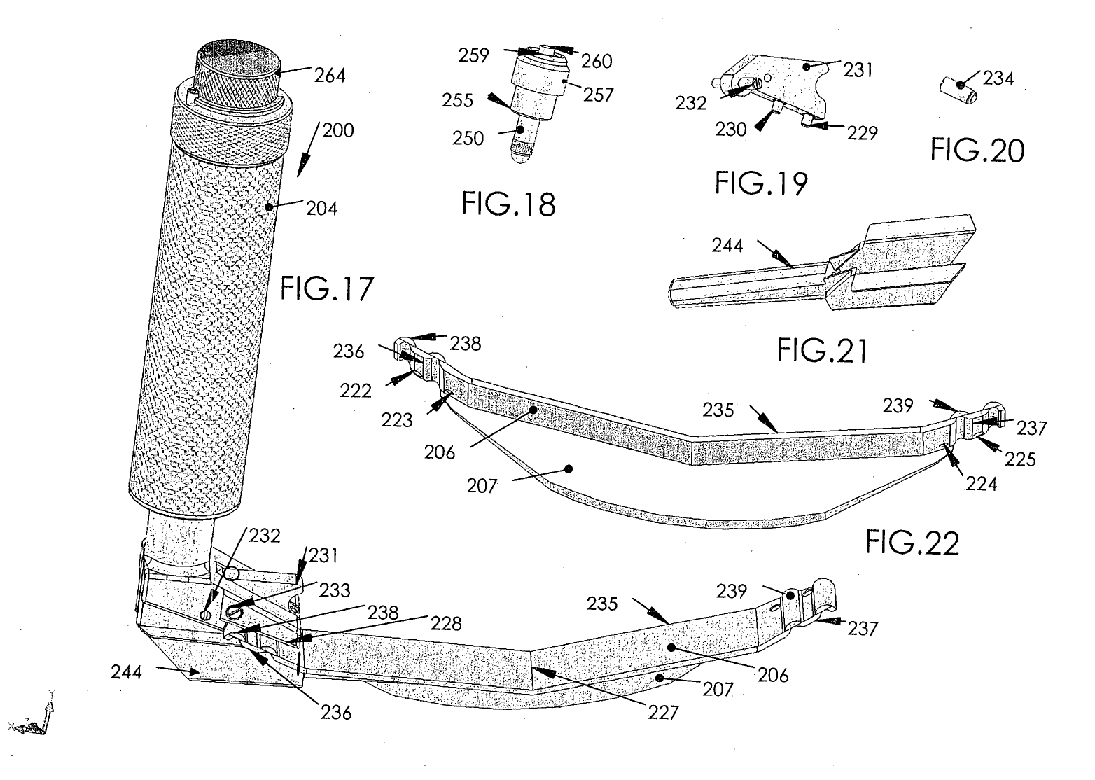

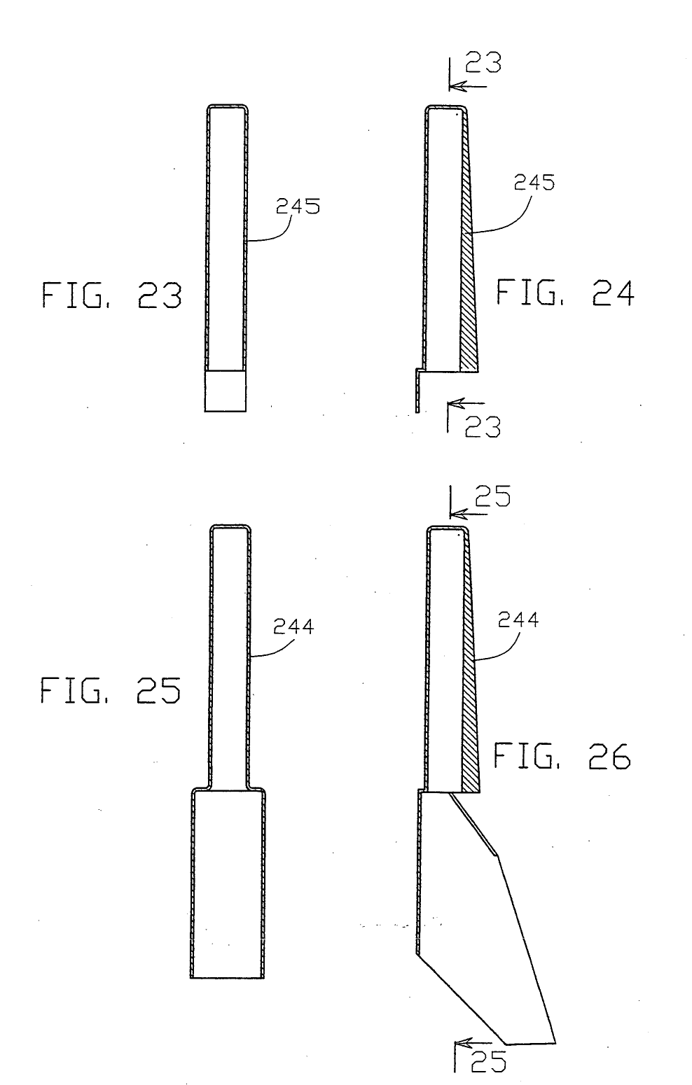

The explanation of the present invention is offered with references made to the attached drawings in FIGS. 6 to 47.

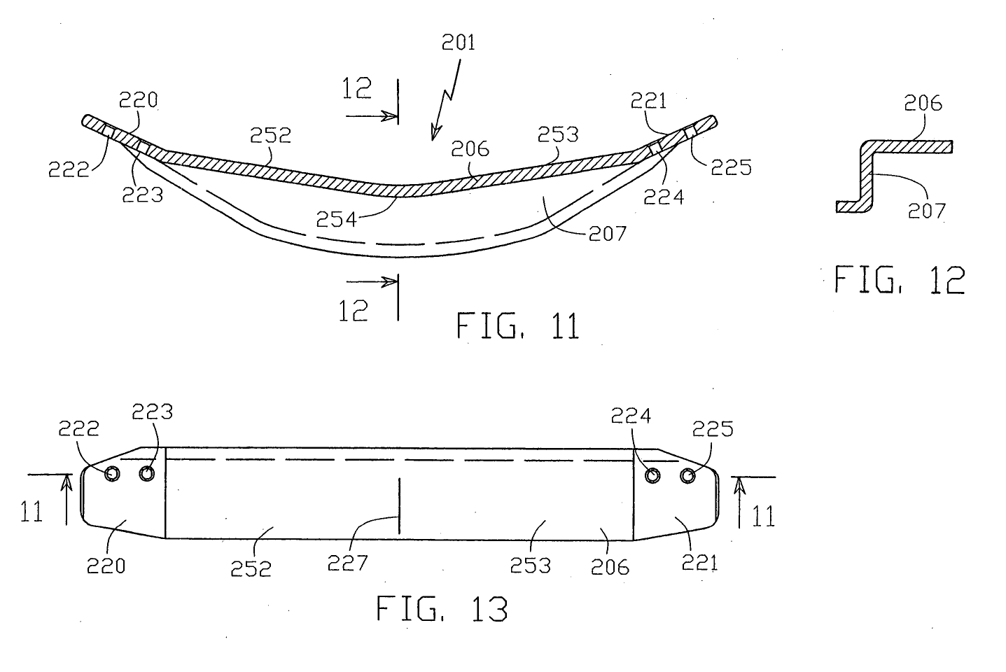

The drawings in FIGS. 6 to 16 show intubation laryngoscope 200 comprising handle 204 and detachable blade 201. The latter, shown as a separate detail in FIGS. 11-13 and 15, has blade distal end portion 202 designed to expose a tracheal entrance in order to insert an endothracheal tube therein, blade proximal end portion 203, upper longitudinal part 206 to be interacted with patient's tongue and lateral longitudinal part 207 disposed laterally and below relative to upper longitudinal part 206. Blade longitudinal parts 206, 207 have the form of a thin-walled sheet of various outlines and curvature. The position of lateral longitudinal part 207 determines what operator's hand, right or left, is used for inserting the endotracheal tube, while another operator's hand holds said handle. Specifically, the disposition of lateral longitudinal part 207 on the left side of upper longitudinal part 206 (see FIG. 14) provides for using the operator's right hand for the endotracheal tube insertion, and conversely the disposition of lateral longitudinal part 207 on the right side of blade upper longitudinal part 206 (see FIGS. 6-8 and 16) provides for using the operator's left hand for the endotracheal tube insertion. The noted left and right sides are determined from operator's point of view. Therewith, each of blade end portions 202 and 203 is adapted for using as blade distal end portion 202 for immediate exposing the tracheal entrance. Therefore, the blade design provides for the capability of previous turning blade 201 through 180° to thereby convert its proximal end portion 203 into distal end portion 202 and simultaneously to change the lateral disposition of lateral longitudinal part 207 relative to upper longitudinal part 206 in order to change the operator's hand to be used for inserting the endotracheal tube. This capability is illustrated with FIGS. 14-16, where blade 201 mounted for the right-handed operation in FIG. 14 is converted into the blade 201 in FIG. 16 designed for the left-handed operation as a result of turning blade 201 through 180°. This capability also is provided with the design of a laryngoscope fastening means, which is capable of detachable connecting blade 201 to handle 204 regardless of what end portion of blade is chosen as the blade distal end portion. Also the laryngoscope illumination means allows illuminating the zone of the endotracheal tube insertion regardless of what blade end portion is chosen as the blade distal end portion. Thus, the capability of both right-handed and left-handed operation of the laryngoscope, depending on operator's choice, is achieved. Therewith it should be noted, the necessity of the offered designs of the blades, fastening means and illumination means for providing the above noted two-handed operation is the cogent argument in support of the invention unity, in spite of the capability of consideration of them as the independent inventions applicable also for another purposes.

The fastening means includes blade fastened sections 220, 221 in the form of the thin-walled sheet portions located on blade 201 and blade engagement elements 222-225. The latter are immovable relative to blade 201 and made as recesses, specifically as openings in blade fastened sections 220, 221. Blade fastened sections 220, 221 and blade engagement elements 222-225 are made of the thin-walled sheet of blade upper longitudinal part 206 and located in the limits of blade end portions 202, 203 so that each of these blade end portions is capable of applying both as blade distal end portion 202 designed for immediate exposing the tracheal entrance and as blade proximal end portion 203 designed for connecting to handle 204 through blade holder 226. Such design of blade engagement elements 222-225 allows unobstructed introducing blade 201 into patient's mouth, normal functioning blade end portions 202, 203 as a means for the exposure of tracheal entrance, and unobstructed inserting the endotracheal tube into trachea. In version embodiments, shown in FIGS. 11-13 and 15, blade 201 is symmetric relative to a mid plane intersecting blade 201 and passing through blade mid line 227.

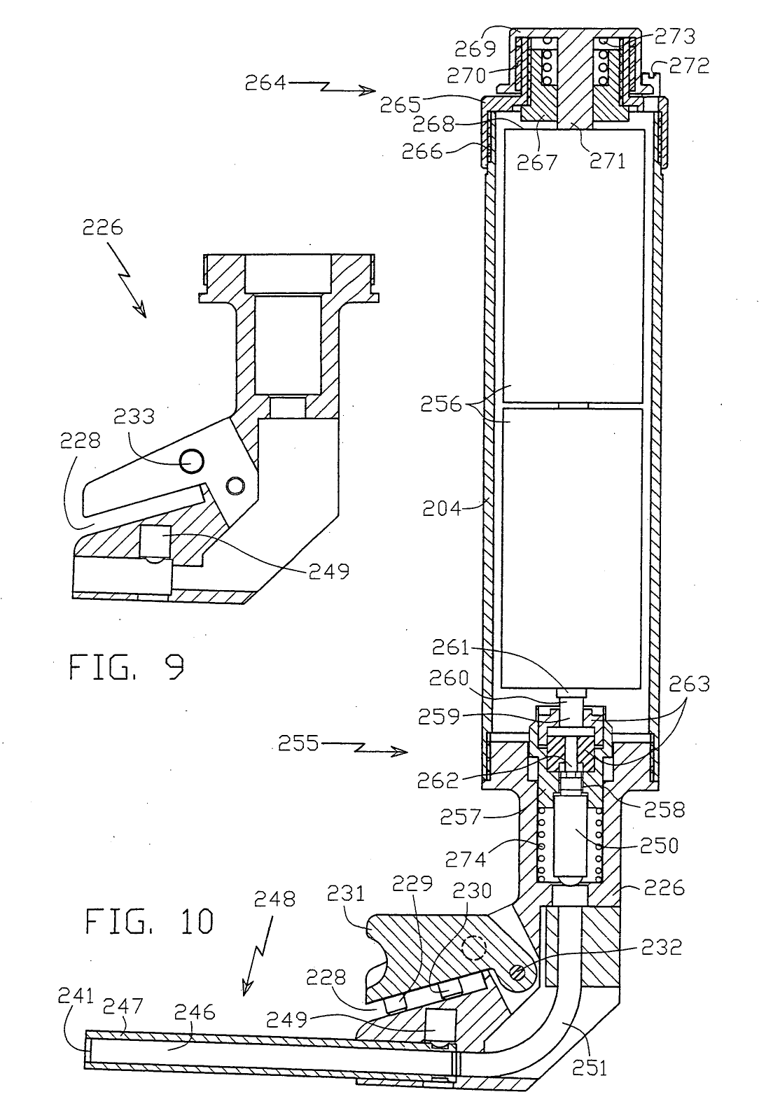

The fastening means also includes blade holder 226 (see FIGS. 9, 10) disposed at the distal end of handle 204, holder slit 228 adapted to inserting thin-walled sheet blade fastened section 220, 221 therein with an insignificant gap to partial fix blade 201 in blade holder 226. Therewith, blade fastened sections 220, 221 are straight and flat, and holder slit 228 is formed with two flat surfaces. There are holder engagement elements 229, 230 made as movable projections. The latter are movable relative to blade holder 226 and capable of entering into engagement with blade engagement element 222, 223 or 224, 225 to fix blade fastened sections 220 or 221 in holder slit 228 and to thereby attach blade 201 to blade holder 226 and handle 204, as well as capable of coming out from the engagement with blade engagement elements 222-225 to decouple blade fastened section 220 or 221 and holder 226 and to thereby detach blade 201 from blade holder 226 and handle 204. Movable engaging projections 229, 230 are located on movable control member 231 made as a lever rotatable around lever axle 232 mounted in blade holder 226, controlled by operator and provided with a fixing means 233 (see FIGS. 9, 14, 16, 19) to fix control lever 231, when blade 201 is attached to blade holder 226. Fixing means 233 present two springy ball stoppers similar to stopper 234 shown in FIG. 20.

The arrangement of the mentioned components of the fastening means is so that, when blade fastened section, for example 221 (see FIGS. 6, 8), is inserted into holder slit 228, movable engaging projections 229, 230 enters engaging openings 224, 225, respectively, thereby fixing blade fastened section 221 in holder slit 228 and attaching blade 201 to holder 226 and handle 204.

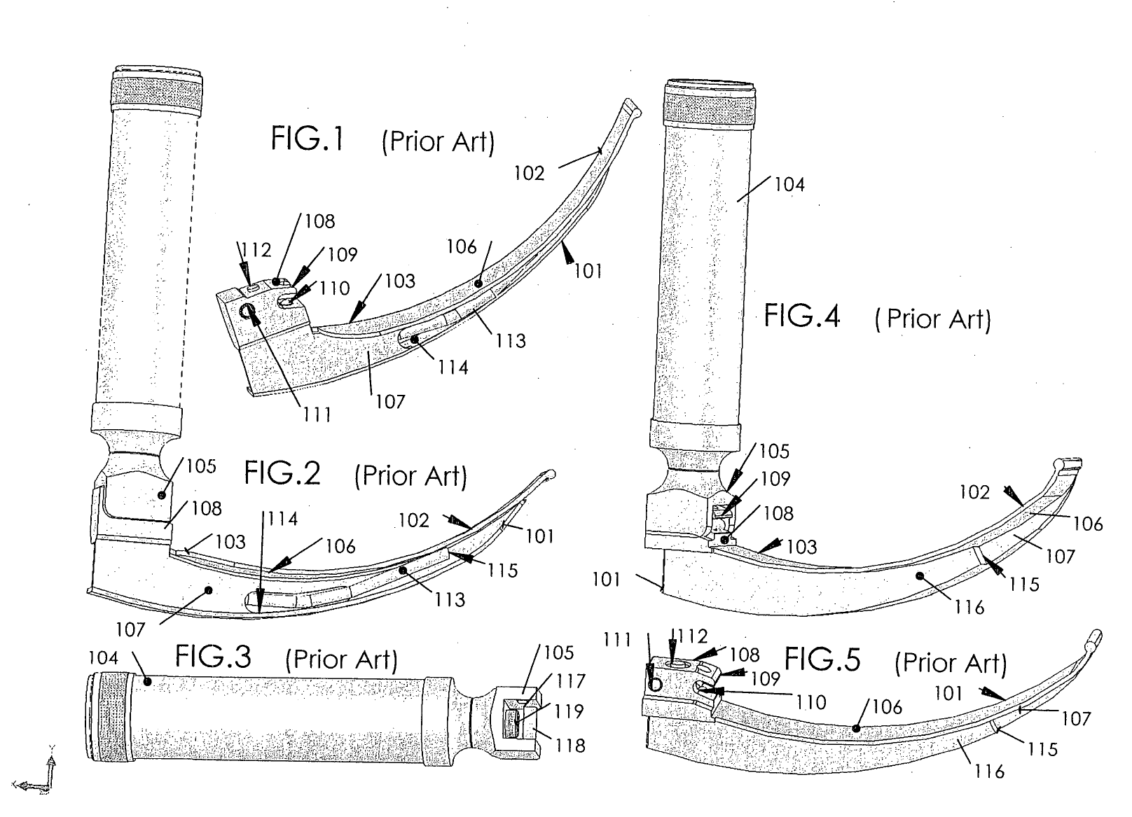

As a result, as distinct from the prior art, the fastening means components, located on the blade, consist only of the thin-walled sheet blade fastened section, which presents the segment of the blade longitudinal part, and the openings thereon. Therewith, the fixing movable engagement elements are disposed only on the blade holder allowing at least fourfold decrease in the number of the fixing movable engagement elements in comparison with the Prior Art (see the laryngoscopes of “Welsh Allyn” company in FIG. 1 and of “Heine” company in FIG. 4), where these elements in the form of springy ball stoppers 110, 111 are located on each of the four blades of the laryngoscope set. Thus, the substantial simplifying the design and reducing the cost of the laryngoscope fastening means is achieved.

In version embodiment (not shown), each of the movable engaging projections presents a ball of a springy ball stopper, which enters the engaging opening under the action of the stopper spring and comes out from the engaging opening overcoming the spring resistance as a result of trailing the blade by operator.

In another version embodiment (not shown), external surface of the blade fastened section is used as the blade engagement element, the holder engagement element presents a clamping member, which, in the blade attached state, presses the external surface of blade fastened section against opposite wall of the holder slit thereby fixing the blade in the holder by means of friction force arising between the external surface of blade fastened section and the holder slit wall.

As a rule, the laryngoscope comprises the set of the detachable blades of different length including blades for children and adults, and each of the detachable blades must be provided with the offered fastening means. So, FIG. 8 shows the laryngoscope with the longest blade 201, while FIG. 6 shows the laryngoscope fastening means as applied to the shortest blade of the blade set. In both these cases, the same fastening means is used.

In version embodiment, holder slit 228 is capable of receiving the thin-walled sheet blade fastened sections of various thickness, for example both reusable metal blade 235, whose sheet thickness amounts in the order of about 1.5 mm, and disposable plastic blade 201, whose sheet thickness amounts in the order of about 3 mm. In this case, the size of holder slit 228 is designed for receiving the thicker fastened section of plastic blade 201, while the fastened sections 236, 237 of metal blade 235 (see FIGS. 7, 17, 22) comprise local stamped convexities 238, 239 providing the total thickness of fastened sections 236, 237 equal to the thickness of plastic blade fastened sections 220, 221. Thus, the certain universality of the offered laryngoscope is achieved.

The offered illumination means of laryngoscope 200 is constantly connected with handle 204 and detachable from blades 201 along with the handle, facilitating the mentioned unobstructed previous turning blade 201 through 180° and providing the needed illumination of the zone of endotracheal tube insertion for any of the blade end portions chosen for using as the distal end portion.

The absence of any connection with the illumination means along with the above simplification of the fastening means elements located on the blade allow considerable simplifying the blade design and reducing by several times the blade cost in the comparison with the prior art.

Moreover, this allows excluding the hard accessible gaps and pockets in the blade design thereby easing and cheapening the procedure of the blade washing and disinfection.

The illumination means include single light extender 240 (see FIGS. 6-8) having distal end 241 emitting the illumination light, mounted in blade holder 226, extended from the blade holder in a distal direction and located below blade upper longitudinal part 206 in the proximity to blade upper longitudinal part 206 and blade lateral longitudinal part 207. In FIGS. 6-8, light extender 240 is made as a fiberoptic light guide mounted in blade holder 226. In version embodiment (not shown), the light extender may be performed as a current-carrying guide with an illuminating lamp disposed at its distal end. The light extender is adapted for operation with any blade of the detachable blade set comprising the detachable blades of various lengths including the shortest blade shown in FIG. 6 for children and the longest blade for adults shown in FIGS. 7, 8. Therewith, the length of the distal extension of light extender 240 from blade holder 226 is chosen as a maximal permissible for normal operation with a shortest blade shown in FIG. 6. Specifically, it means that the distance between extender distal end 241 and blade distal end 242 must be sufficient for placing epiglottis and light extender distal end 241. Maximal permissible length of single light extender 240 is, when it extends distally up to the shortest blade distal end but not protrude distally beyond the shortest blade distal end. Minimal expedient length of single light extender 240 is, when it extends distally from the blade holder to at least 15% of a longest blade length. Such illumination means, in comparison with the prior art (see Patent Application US 2005/0159649 A1), does not demand so considerable increase of the light power, is compatible with out-of-straight blades and is not shaded with the endotracheal tube. Light extender 240 is provided with strengthened sheath 243 made of metal, located beneath blade upper longitudinal part 206 adjacent to it, providing a reliable mechanical protection of light extender 240 and allowing using strengthened sheath 243 as a support of the blade resulting in substantial decrease of the requirements for blade strength and the corresponding decrease of the thickness of blade walls and blade weight. This allows using the inexpensive and easy processed plastic for fabricating the disposable blades of relatively simple design shown in FIGS. 6, 8, 11-13, 15. Eventually, this leads to additional considerable reducing the blade cost resulting in throughout extension of the use of disposable plastic blades due to the drastic reduction of the blade cost and heightening the plastic blade reliability due to prevention of the blade breakdowns. Strengthened sheath 243 is covered with transparent plastic protector 244 shown in FIGS. 21, 25, 26 as a separate detail, preventing light extender 240 and strengthened sheath 243 from infecting in patient's mouth and used as a disposable part. Plastic protector 244 allows to cover not only light extender 240 and strengthened sheath 243 inserted into patient's mouth, but also the blade holder walls located beneath blade 201. In version embodiment shown in FIGS. 6, 23, 24, plastic protector 245 is designed to protect only the mentioned parts inserted into patient's mouth.

In version embodiment (see FIG. 10), light extender 246 and strengthened sheath 247 are made as unit 248 detachably fastened to blade holder 226 by means of springy ball stopper 249 allowing detaching unit 248 from the blade holder to sterilize it after infecting in patient's mouth. Therewith, the light is transmitted to light extender 246 from illumination lamp 250 through upper light guide 251. In this case, the offered laryngoscope can be used without plastic protectors 244, 245 as a fully reusable device including reusable metal blades, maintaining the laryngoscope two-handed operation and the mentioned simplicity of the blade design.

Blade upper longitudinal part 206 consists of four variously inclined straight segments forming a blade profile approximately similar to Macintosh profile, including two relatively short peripheral segments 220, 221, distal and proximal, adapted for immediate exposing the tracheal entrance and for using them as the blade fastened section, as well as two internal segments 252, 253 adapted for interacting with patient's tongue, for favorable arrangement of light extender 240 thereunder and for passing the illumination light from light extender distal face 241 to the zone of endotracheal tube insertion located beneath the peripheral distal segment (see FIGS. 11, 13, 14, 15). Therewith, the straightness of peripheral segments 220, 221 is needed for their easy insertion into holder slit 228, and the straightness of internal segments 252, 253 is needed for passing the illumination light from light extender distal face 241 to the zone of endotracheal tube insertion, and, as applied to the longest blade of the blade set, the light extender distal face 241 is located near a transient zone 254 between two internal segments 252, 253 (see FIG. 8).

The offered fastening means does not allow using the light switch turning on the illumination lamp by the blade, which is used in the Prior Art, specifically in the laryngoscopes of Welsh Allyn and Heine companies. In this connection, the present illumination means comprises the new light switch design shown in close-up view of FIG. 10. The present illumination means comprises lamp cartridge 255 (see also FIG. 18) located movably beneath batteries 256 and including cartridge housing 257 having inner thread 258 in its lower portion for screwing in illumination lamp 250. Cartridge contactor 259 is housed within cartridge housing 257 and has upper end 260 to be constantly contacted with lower electrical contact 261 of batteries 256, and lower end 262 to be constantly contacted with lamp 250. Cartridge contactor 259 is insulated from cartridge housing 257 by cartridge insulators 263 housed inside cartridge housing 257. The illumination means includes light switch 264 located at the upper end of handle 204 and allowing operator to turn on and turn off illumination lamp 250. Light witch 264 comprises: switch housing 265 located above batteries 256, made as a handle cap and connected to the handle upper end by means of thread 266; switch insulator 267 housed inside switch housing 265, protruding downward from the switch housing and to be contacted with upper electric contact 268 of batteries 256, when illumination lamp 250 is turned off; switch contactor 269 rotatably mounted on switch housing 265 by means of thread 270, having the constant electric contact with illumination lamp 250 through switch housing 265, metal handle 204 and cartridge housing 257, and comprising contacting protrusion 271 capable of axial moving within switch insulator 267 as a result of rotating switch contactor 269. Light switch 264 is provided with limiter 272 of said switch contactor rotation mounted on switch housing 265 and determining the angular position “On” and “Off” of switch contactor 269. During moving contacting protrusion 271 downward, it comes into contact with batteries upper electric contact 268 thereby connecting the batteries upper electric contact with illumination lamp 250 and turning on the latter. During moving contacting protrusion 271 upward it comes out from contact with batteries upper electric contact 268 thereby disconnecting the batteries upper electric contact and illumination lamp and turning off the latter. Cartridge insulators 263 and switch insulator 267 are made of Teflon whose temperature properties allow autoclaving the laryngoscope. In version embodiment, switch contactor 269 is loaded with upper spring 273 to prevent switch contactor 269 from spontaneous rotation, when illumination lamp 250 is turned off.

Lower spring 274 is disposed between cartridge housing 257 and blade holder 226 and designed to provide the reliable constant electric contact between cartridge contactor upper end 260 and batteries lower electrical contact 261, as well as between batteries upper electric contact 268 and contacting protrusion 271 of switch contactor 269, when illumination lamp is turned on. Besides, lower spring 274 serves as a lower support of batteries 256 and lamp cartridge 255 providing their reliable mounting in laryngoscope. Thus, the present light switch has no any connection with the laryngoscope blades allowing the unobstructed turning on and turning off the illumination light independently of the blade position. As distinct from the Prior Art, the present light switch may be controlled by the same operator's hand, which holds the handle. The present lamp cartridge 255 along with lamp switch 264 comprise substantially less the part number than the lamp cartridge assembly of Prior Art and these parts demand less the fabrication accuracy. As a result, the noted components of the present laryngoscope are cheaper. As distinct from the Prior Art, the present lamp cartridge 255 has no mutually movable parts and is fully sealed thereby preventing the parts from jamming and from getting a liquid inward during the lamp cartridge disinfecting.

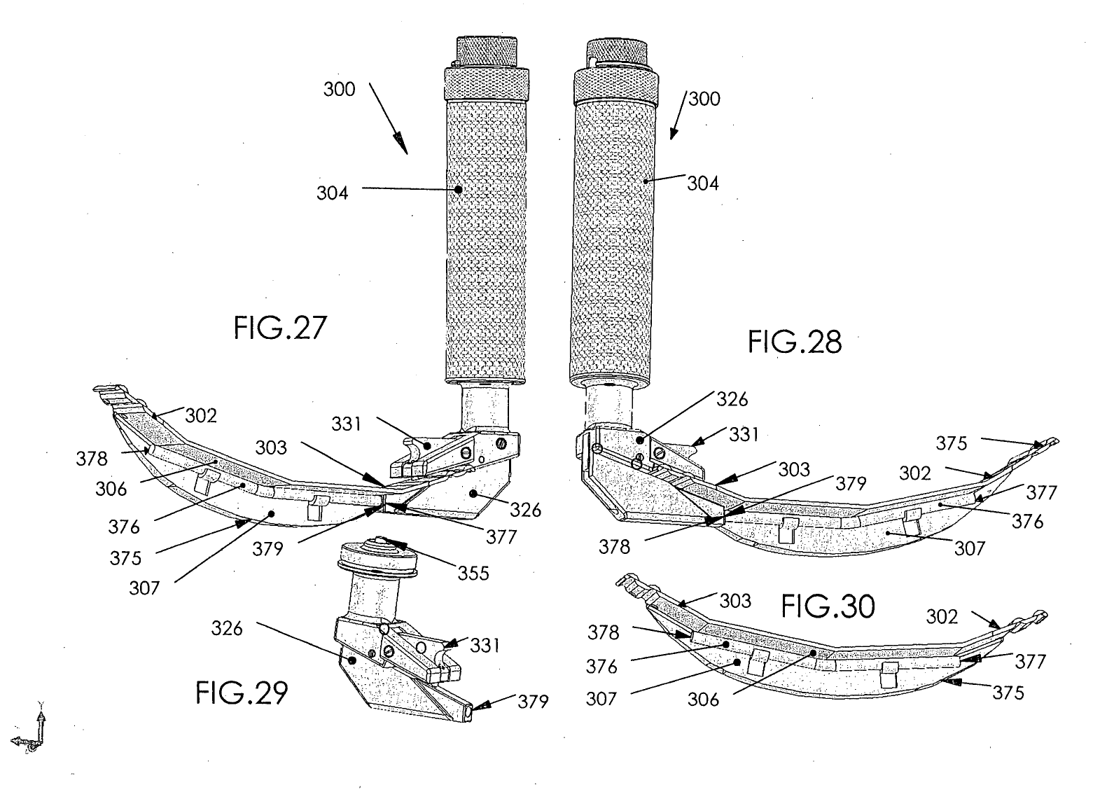

In version embodiment (see FIGS. 27 to 30), laryngoscope 300 comprises the illumination means including one transmitting light guide 376 mounted along reusable metal blade 375 adjacent to blade upper longitudinal part 306 and lateral longitudinal part 307, having transmitting light guide distal end 377 located proximally of blade end distal portion 302 and transmitting light guide proximal end 378 located distally of blade end proximal portion 303 in front of distal light emitting surface 379 of blade holder 326 (see FIGS. 28-30). As a result, the light is transmitted from holder light emitting surface 379 through transmitting light guide 376 to the zone of endotracheal tube insertion. Therewith, turning blade 375 through 180° leads to converting transmitting light guide distal end 377 into the transmitting light guide proximal end 377 (see FIG. 27) providing the same light transmission from holder light emitting surface 379 to the zone of endotracheal tube insertion. This version also allows maintaining the laryngoscope two-handed operation in the fully reusable laryngoscope.

In another version embodiment (not shown), every blade has two transmitting light guides, which are mounted along the blade adjacent to the blade upper longitudinal part and the blade lateral longitudinal part, and each of these two transmitting light guides is designed for operation with one certain blade end portion providing the light transmission from the holder distal light emitting surface to the zone of endotracheal tube insertion. Turning the blade through 180° leads to putting into operation another transmitting light guide providing the same light transmission from the holder distal light emitting surface to the zone of the endotracheal tube insertion. Therewith, the advantage of this version is the capability of choosing the optimal distance between the transmitting light guide distal ends and the blade distal end.

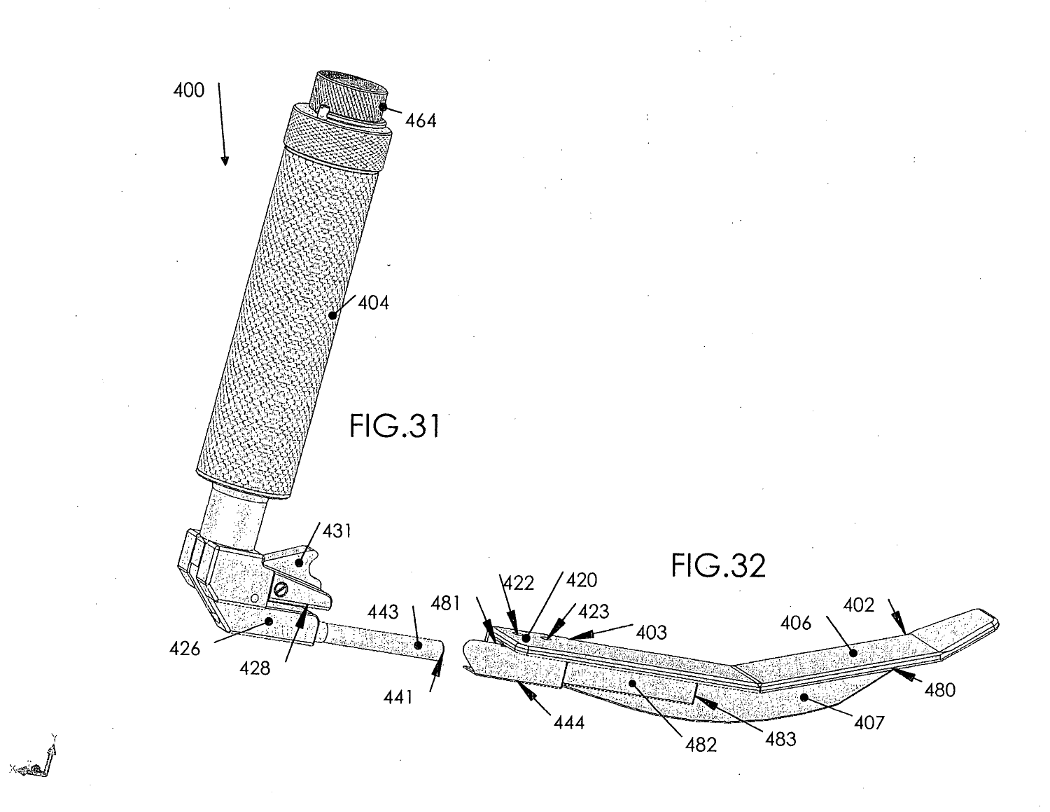

FIGS. 31, 32 show laryngoscope version 400, having the illumination means, which in many respects is similar to shown in FIGS. 8, 14. Specifically, it has the same light extender and strengthened sheath 443 mounted in blade holder 426. However, plastic protector 444 of the light extender and strengthened sheath is made as an integral part of blade 480, as distinct from detachable plastic protector 244 (see FIG. 21), used in laryngoscopes 200. Plastic protector 444 includes blade socket 481 located at blade proximal end portion 403, opened from a proximal side of the blade, and having distal extension 482 designed to hose the light extender and strengthened sheath in order to insulate them from patient's mouth medium. Therewith, blade socket 481 includes the proximal lower wall and the proximal lateral wall completely insulating blade holder 426 from any contact with an endotracheal tube during the intubation procedure to prevent the blade holder from infecting. Socket distal extension 482 has transparent distal face 483 located in front of light extender distal end 441 emitting the illumination light. In version embodiment, blade 480 as a whole is made of transparent plastic. The fabrication of the blade and anti-infection protector as a single part allows easing the attachment/detachment of the blade to/from the laryngoscope, decreases the part number, and is convenient for using the blade as a disposable component. However, the arrangement of socket 481 at blade proximal end portion 403 does not allow using blade 480 for the two-handed operations. Otherwise, the design and operation of laryngoscope 480 is similar to one of above laryngoscope 200.

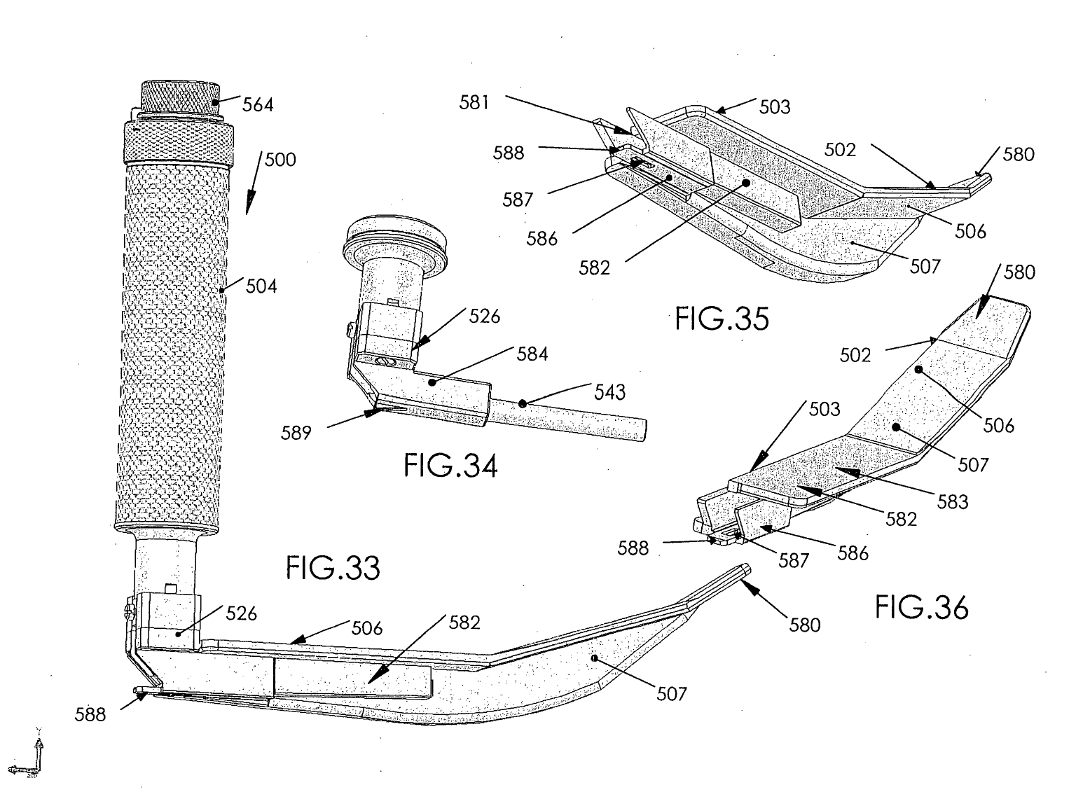

However, the anti-infection protector made as a blade proximal socket may be used not only as the anti-infection means shown above in FIGS. 31, 32, but also as a constituent of a fastening means. Such functions are inherent in blade proximal socket 581 of laryngoscope 500 shown in FIGS. 33 to 36. The fastening means of laryngoscope 500 includes blade socket 581, fastening protrusion 584 of blade holder 526 protruding distally of blade holder 526, adapted to inserting into blade socket 581 and having the external configuration corresponding to the inner configuration of blade socket 581 to maximally restrict the mutual displacement of blade holder 526 and blade 580 after inserting the fastening holder protrusion 584 into blade socket 581. The fastening means also has the engagement appliance designed to eliminate the mutual displacement of blade holder 526 and blade 580 after inserting holder protrusion 584 into bade socket 581. The engagement appliance is made as a latch consisting of: flexible leg 586 made of the blade socket lower wall and including window 587 disposed in the leg proximal portion. There is also holder projection 589 located on the external lower wall of holder protrusion 584 and adapted for automatic entering leg window 587 during the end stage of inserting holder protrusion 584 into blade socket 581 to thereby fix holder protrusion 584 in blade socket 581. Therewith, in the fixed position, flexible leg proximal end 588 protrudes proximally of blade holder 526 and is accessible for operator's pressing downward to disengage flexible leg 586 and holder projection 589 and to detach blade 580 from blade holder 526. At the same time, blade socket 581 has distal extension 582 to hose therein strengthened sheath 543 along with the light extender for the anti-infection protection of the illumination means. The blade 580 is designed preferably for the disposable use because the socket 581 and its distal extension 582 are hard accessible for washing.

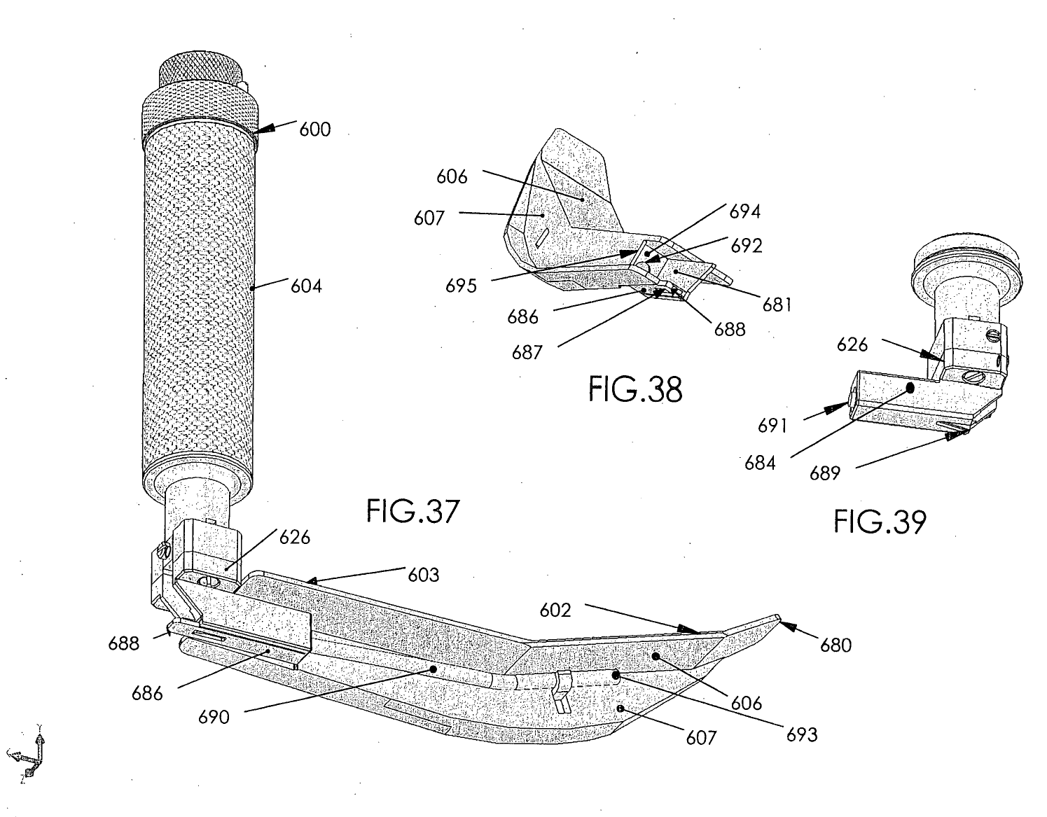

The laryngoscope 600 with the reusable version of the blade having the blade socket as a fastening means is shown in FIGS. 37 to 39. The illumination means of laryngoscope 600 includes transmitting light guide 690 mounted on blade 680, disposed along the blade adjacent to blade upper longitudinal part 606 and lateral longitudinal part 607, having transmitting light guide distal end 693 located proximally of blade end distal portion 602 and transmitting light guide proximal end 692 located inside blade socket 681 at distal wall 694 of blade socket 681 in front of distal light emitting surface 691 of holder protrusion 684, when the latter is inserted into blade socket 681. Blade socket 681 is partly opened from the left side due to using shortened socket left wall 695. Partly opened socket 681 and the absence of the socket distal extension ease washing the blade 680 allowing its reusable use. Otherwise, the design and operation of laryngoscope 600 is identical to one of above laryngoscope 500.

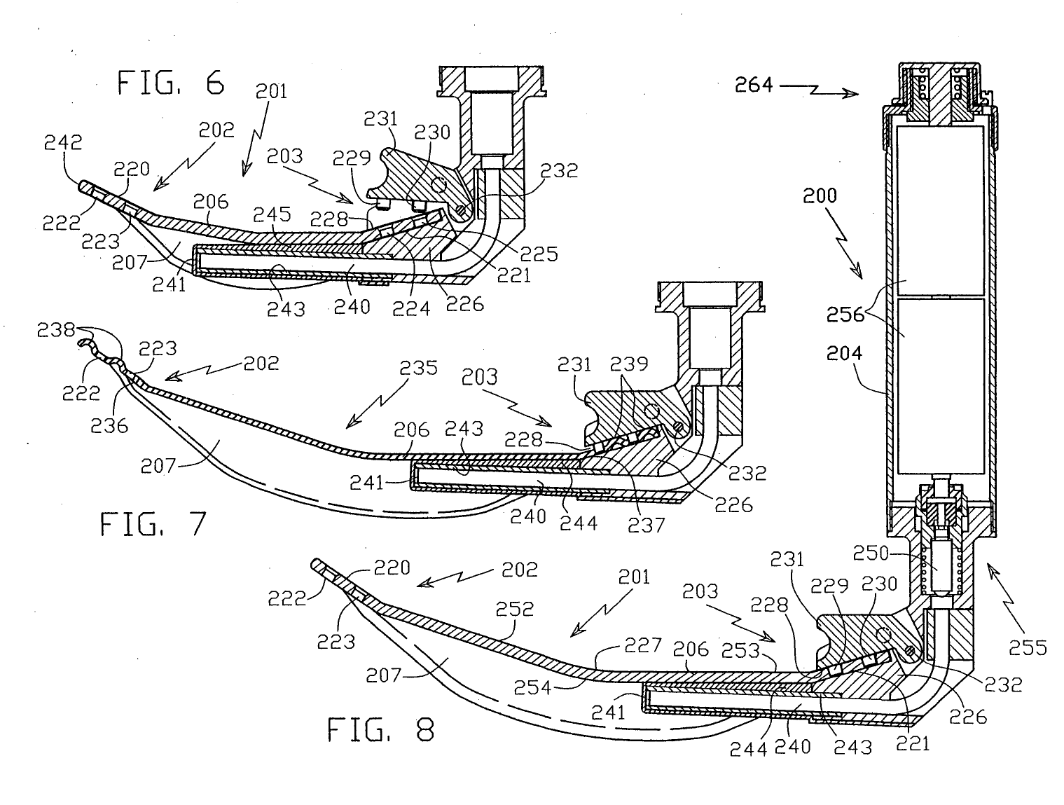

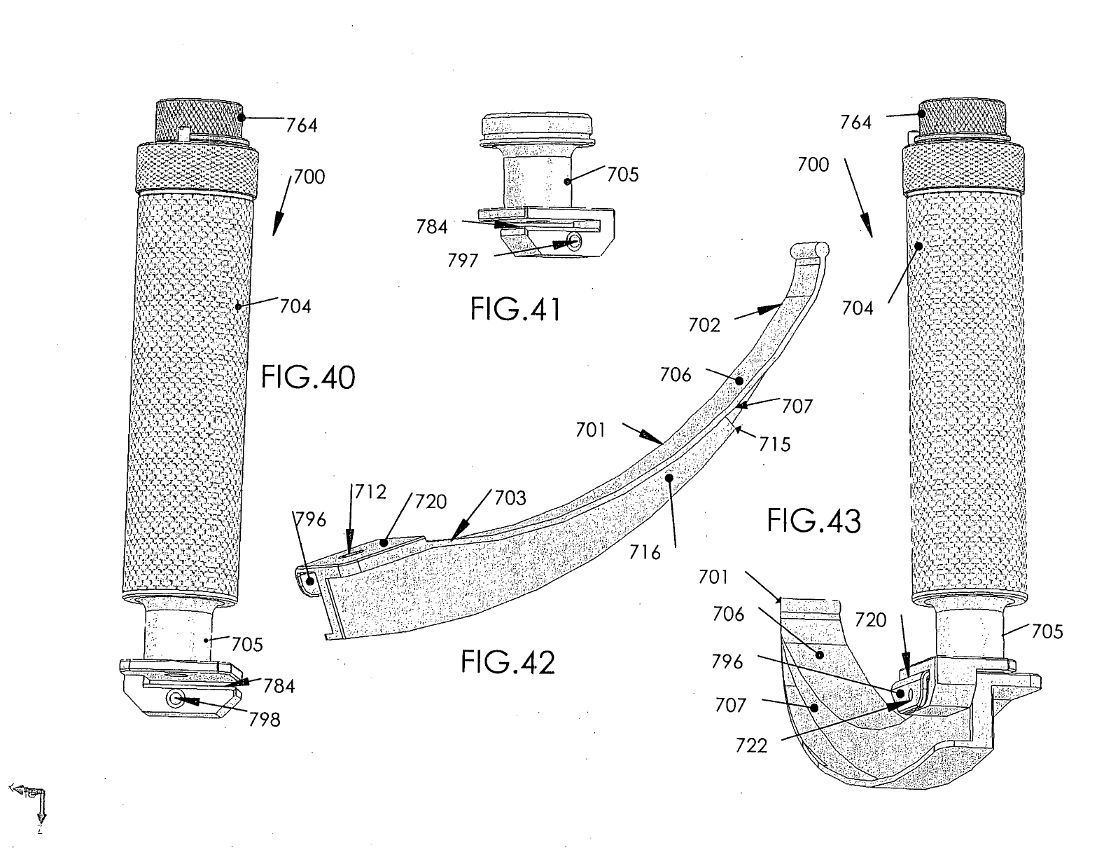

The present fastening means needed for obtaining the above two-handed operation of the laryngoscope blade is applicable also for other purposes. Specifically, FIGS. 40 to 43 show laryngoscope 700, wherein the offered fastening means is used in the combination with the existing reusable blade of Heine's company (see FIGS. 4, 5) instead of existing fastening means. The offered fastening means includes: blade fastened section 720 in the form of a thin-walled sheet portion located on blade 701; blade engagement element 722 disposed at blade fastened section 720, immovable relative to blade 701 and made in the thin-walled sheet as an opening in the limits of blade proximal end portion 703; blade holder 705 disposed at the distal end of handle 704 and including holder slit 784 adapted to inserting thin-walled sheet blade fastened section 720 therein with an insignificant gap to partial fix blade 701 in blade holder 705. Holder engagement elements present balls 797, 798 of two-sided springy ball stopper movable relative to blade holder 705, which enter opening 722 and opening (not shown) oppositely disposed in wall 707 under the action of the stopper spring to thereby attach blade 701 to blade holder 705 and handle 704 and come out from opening 722 and the oppositely disposed opening overcoming a spring resistance as a result of trailing the blade 701 by operator to thereby detach blade 701 from blade holder 705 and handle 704. The fastening means includes lateral restrictor 796 of the lateral movement of blade fastened section 720 in holder slit 784 made as the bended continuation of blade fastened section 720, which in the blade attached state is disposed beyond holder slit 784 adjacent to it. The illumination means of laryngoscope 700 includes the fiberoptic light guide disposed in closed sheath 716 of blade 701 similar to one of blade 101 of company “Heine” (see FIGS. 4, 5) as well as lamp cartridge 255 and light switch 264 shown in FIG. 10.

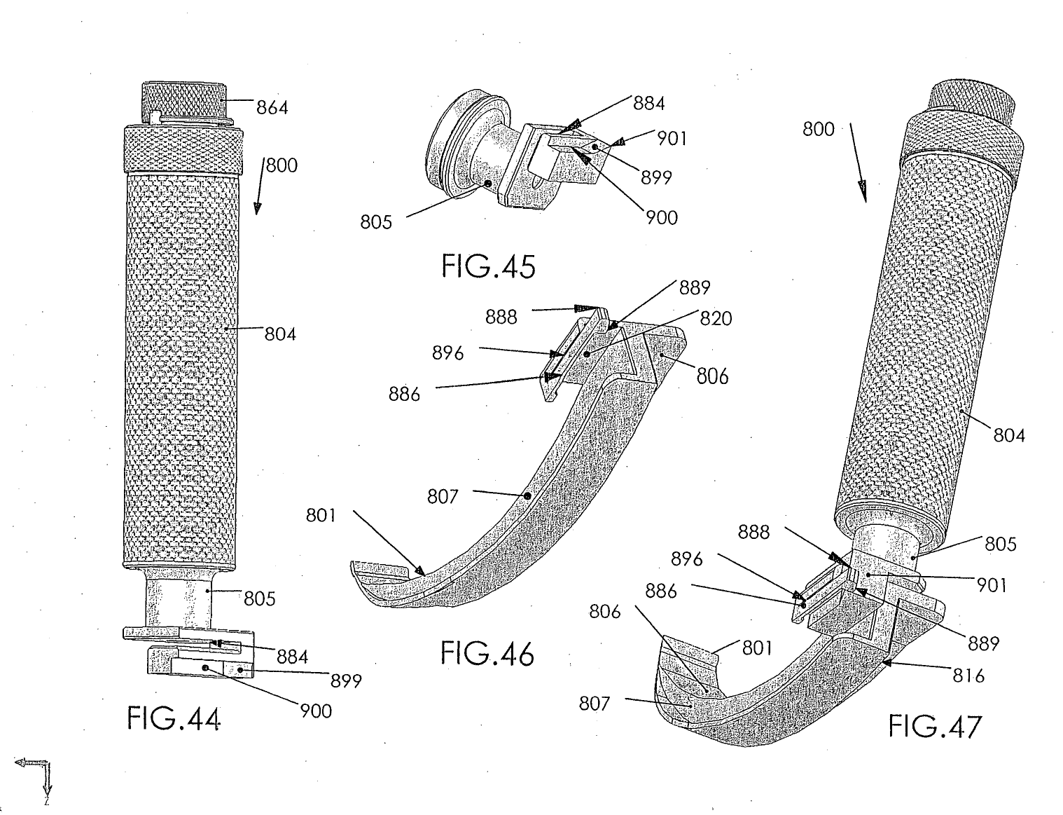

FIGS. 44 to 47 show laryngoscope 800, wherein thin-walled blade fastened section 820 of detachable blade 801 is fixed in holder slit 884 by means of engagement appliance made as the latch. The latter comprises resilient leg 886 located on blade 801 and having blade engagement element 889 movable relative to blade 801 and interacting with holder engagement element 901, which presents the back flat surface of blade holder 805 immovable relative to the blade holder. To attach blade 801 to blade holder 805 thin-walled blade fastened section 820 is inserted into holder slit 884. In doing so, blade engagement element 889 free moves in recess 900 of blade holder 805 and then slides on inclined portion 899 of the blade holder deviating laterally from blade holder 805 up to entering into engagement with holder engagement element 901 as it is shown in FIG. 47. To detach blade 801 from blade holder 805 operator presses laterally proximal end 888 of resilient leg 886 thereby disengaging blade engagement element 889 from holder engagement element 901 and withdraws blade fastened section 820 from holder slit 884. Otherwise, the design and operation of laryngoscope 800 is identical to one of laryngoscope 700.

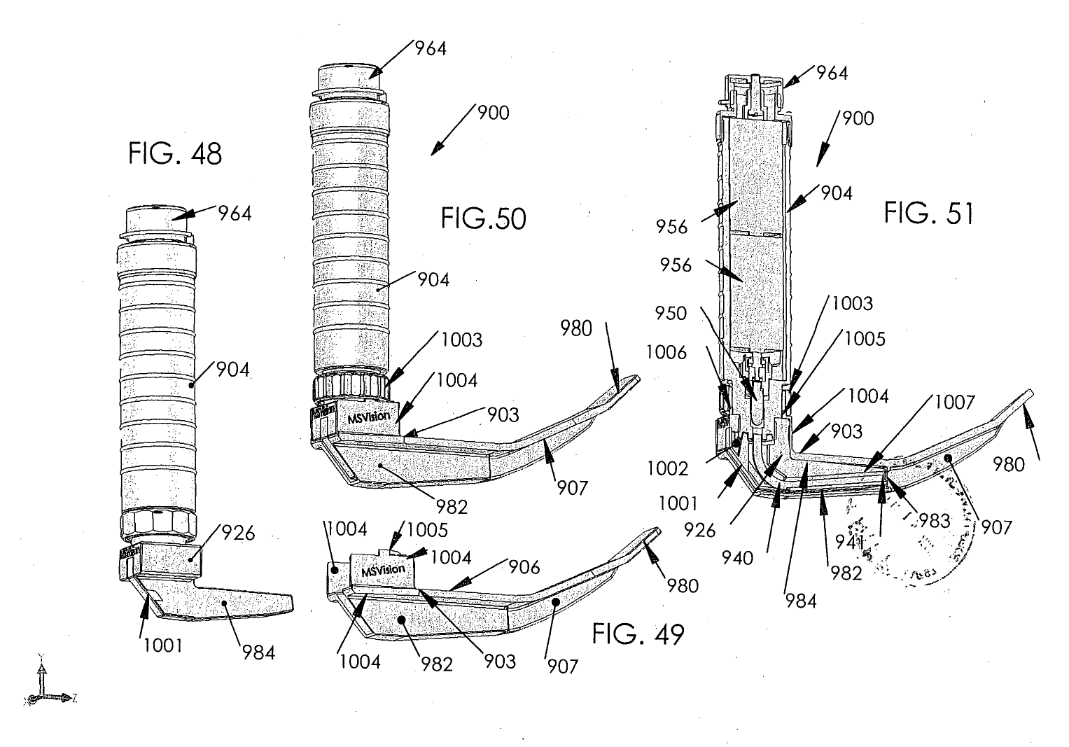

FIGS. 48 to 51 show laryngoscope 900, which is the version embodiment of laryngoscope 500 shown in FIGS. 33 to 36. As distinct from laryngoscope 500, version 900 has blade socket 982, which serves as a component of the fastening means capable of detachable connecting blade 980 and handle 904 through blade holder 926. The latter is provided with fastening protrusion 984 made as an integral part of blade holder 926, protruding distally of blade holder 926, adapted to inserting into blade socket 982 and having external configuration corresponding to an inner configuration of blade socket 982 to maximally restrict the mutual displacement of blade holder 926 and blade 980 after inserting fastening protrusion 984 into blade socket 982. The engagement appliance of laryngoscope 900 includes fastening nut 1003 movably connected by thread with the lower portion of handle 904, and arcuated ridge 1005 located on an upper surface of blade proximal end portion 903 and designed to enter circular recess 1006 in a lower face of fastening nut 1003 to engage blade 980 and blade holder 926. This engagement appliance is designed for detachable engagement of blade holder 926 and blade 980, when fastening protrusion 984 is inserted into blade socket 982. To disengage them fastening nut 1003 should unscrew upward. Single light extender 940 in the form of fiberoptic light guide is disposed within channel 1007 of fastening protrusion 984 so that the distal faces of single light extender 940 and channel 1007 are nearby each to other. Blade holder 926 has rear cavity 1002 for introducing and mounting single light extender 940 therein, and this cavity is provided with detachable lid 1001. Fastening protrusion 984 serves as a strengthened sheath of single light extender 940 and as a support of blade 980 during the intubation procedure. The function of fastening protrusion 984 as the support results in substantial decrease of the requirements for blade strength and the corresponding decrease of the thickness of blade walls and blade weight. Blade socket 982 includes proximally arranged lower, lateral and anterior walls 1004 completely insulating blade holder 926 from any contact with patient and an endotracheal tube during the intubation procedure to prevent the blade holder from infecting.

In version embodiment (not shown), blade holder 926, handle 904 and fastening protrusion 984 are made as an integral part of plastic.

1.-82. (canceled)

83. An intubation laryngoscope comprising
a handle,
at least one detachable blade, having
a blade distal end portion designed to expose a tracheal entrance in order to insert an endothracheal tube therein,
a blade proximal end portion,
an upper longitudinal part,
a lateral longitudinal part disposed laterally and below relative to said upper longitudinal part, and the position of said lateral longitudinal part determines what operator's hand, right or left, is used for inserting said endotracheal tube, while another operator's hand holds said handle, so that the disposition of said lateral longitudinal part on the left side of said upper longitudinal part provides for using the operator's right hand for said endotracheal tube insertion, and conversely the disposition of said lateral longitudinal part on the right side of said blade upper longitudinal part provides for using the operator's left hand for said endotracheal tube insertion, therewith the left and right sides are determined from operator's point of view,
therewith, each of said blade end portions is adapted for using as said blade distal end portion for immediate exposing said tracheal entrance,
and there is a capability of previous turning said blade through 180° to thereby convert its said proximal end portion into said distal end portion and to change the lateral disposition of said lateral longitudinal part relative to said upper longitudinal part in order to change the operator's hand to be used for inserting said endotracheal tube,
a fastening means capable of detachable connecting said blade to said handle regardless of what blade end portion is chosen as said blade distal end portion, including
a blade holder disposed at an end of said handle,
an illumination means designed to illuminate the zone of said endotracheal tube insertion regardless of what blade end portion is chosen as said blade distal end portion, and provided with means for needed turning on and turning off the light, which does not obstruct the normal functioning said fastening means.

84. The intubation laryngoscope of claim 83, wherein
said blade upper longitudinal part has the form of a thin-walled sheet of various outlines and curvature,
said fastening means includes
a blade fastened section in the form of a thin-walled sheet portion,
at least one blade engagement element disposed at said blade fastened section,
and said blade fastened section and said blade engagement element are located in the limits of each of said blade end portions so that each of said blade end portions is capable of applying both as said blade distal end portion designed for immediate exposing said tracheal entrance and as said blade proximal end portion designed for connecting to said handle through said blade holder,
said blade holder includes
a holder slit adapted to inserting said thin-walled sheet blade fastened section therein with an insignificant gap to partial fix said blade in said blade holder,
at least one holder engagement element, adapted to detachable engagement with said blade engagement element.

85. The intubation laryngoscope of claim 84, wherein
said blade engagement element is immovable relative to said blade,
said holder engagement element is movable relative to said blade holder and capable of entering into engagement with said blade engagement element to fix said blade fastened section in said holder slit and to thereby attach said blade to said blade holder and handle, as well as capable of exiting from said engagement with blade engagement element to decouple said blade fastened section and said holder and thereby to detach said blade from said blade holder and handle.

86. The intubation laryngoscope of claim 85, wherein said blade engagement element is made as an engaging opening in said blade fastened section, and said holder engagement element is made as a movable engaging projection located on a movable control member disposed in said blade holder, controlled by operator and provided with a fixing means to fix it, when said blade is attached to said blade holder.

87. The intubation laryngoscope of claim 84, wherein said illumination means is connected with said handle and along with said handle fully detachable from said blades, allowing unobstructed previous turning said blade through 180° and providing the needed illumination of said zone of endotracheal tube insertion for any end portion of said blade chosen for using as said distal end portion.

88. The intubation laryngoscope of claim 87, wherein said illumination means include a single light extender mounted in said blade holder, having a distal end emitting said illumination light, extended from said blade holder in a distal direction, located below said blade upper longitudinal part in the proximity to said blade upper longitudinal part and said blade lateral longitudinal part, and adapted for operation with any blade of detachable blade set including detachable blades of different lengths.

89. An intubation laryngoscope, comprising
a handle,
a set of detachable blades of different length, wherein each of said detachable blades has
a blade distal end portion designed to expose a tracheal entrance in order to insert an endotracheal tube therein,
a blade proximal end portion,
an upper longitudinal part,
a fastening means capable of detachable connecting said blade to said handle, including
a blade holder disposed at an end of said handle,
at least one holder engagement element disposed in said blade holder,
at least one blade engagement element disposed on said each of detachable blades and adapted to detachable engagement with said holder engagement element,
an illumination means, therewith
said illumination means is connected with said blade holder,
said illumination means along with said blade holder are fully detachable from said blades,
the same single said illumination means provides the needed illumination of a zone of endotracheal tube insertion for any blade of said blade set,
said illumination means includes
a single light extender
adapted for operation with any blade of said blade set,
having a distal end emitting said illumination light and extended from said blade holder in a distal direction sufficiently to provide the needed illumination for any blade of said blade set, and
located below said blade upper longitudinal part in the proximity to it.

90. The intubation laryngoscope of claim 89, wherein said distal end of single light extender is disposed in proximity to a distal end of a shortest blade of said detachable blade set, but does not protrude distally relative to said shortest blade distal end and does not hinder said blade distal end from necessary interaction with patient's tissues.

91. The intubation laryngoscope of claim 89, wherein said distal end of single light extender extends distally from said blade holder to at least 15% of a longest blade length.

92. The intubation laryngoscope of claim 89, wherein said single light extender serves as a support of said blade upper longitudinal part thereby decreasing a blade strain and resulting in substantial decrease of the requirements for blade strength and the corresponding decrease of the thickness of blade walls and blade weight, as well as providing the favorable conditions for using inexpensive and easy processed material of blades as plastic.

93. The intubation laryngoscope of claim 92, wherein said single light extender has a strengthened sheath connected to said blade holder, located beneath said blade upper longitudinal part adjacent to it, providing a reliable mechanical protection of said light extender and improving its properties as said blade support.

94. The intubation laryngoscope of claim 92, wherein said single light extender is covered with a transparent plastic protector used as a disposable part and preventing said single light extender from infecting in patient's mouth.

95. The intubation laryngoscope of claim 92, wherein said single light extender is made as a detachable unit allowing its detachment from said blade holder to sterilize after infecting in patient's mouth.

96. The intubation laryngoscope of claim 89, wherein each blade of said blade set is made of transparent plastic, said blade proximal end portion includes a socket designed to hose said single light extender in order to insulate said single light extender from patient's mouth medium, and said blade socket also serves as a part of said fastening means.

97. An intubation laryngoscope comprising
a handle,
a blade holder, located at a lower end of said handle,
a set of detachable blades of different lengths, wherein each of said detachable blades has
a blade distal end portion designed to expose a tracheal entrance in order to insert an endotracheal tube therein,
a blade proximal end portion, designed to connection with said handle through said blade holder,
an upper longitudinal part,
a blade socket located at said blade proximal end portion, opened from a proximal side of said blade,
a fastening means capable of detachable connecting said blade to said handle through said blade holder and including
said blade socket,
a fastening protrusion of said blade holder,
protruding distally of said blade holder,
adapted to detachable inserting into said blade socket,
having external configuration corresponding to an internal configuration of said blade socket to restrict a mutual displacement of said blade holder and said blade after inserting said fastening protrusion into said blade socket,
and serving as a support of said blade during the intubation procedure thereby decreasing the blade strain,
an engagement appliance including engagement elements disposed on said blade proximal end portion and on a unit of said blade holder and said handle lower portion, and said engagement appliance is designed for detachable engagement of said blade holder and blade, when said fastening protrusion is inserted into said blade socket,
an illumination means providing the needed illumination of a zone of endotracheal tube insertion for any blade of said blade set.

98. The intubation laryngoscope of claim 97, wherein said engagement appliance is made as a fastening nut movably connected by thread with said unit of blade holder and handle lower portion and a ridge located on an upper surface of said blade proximal end portion and adapted to entering a circular recess in a lower face of said fastening nut to engage said blade and said blade holder.

99. The intubation laryngoscope of claim 97, wherein said engagement appliance is made as a latch consisting of a resilient leg made as a part of a blade socket lower wall and including a window disposed in a leg proximal portion, and a holder projection located on an external lower surface of said blade holder and adapted to enter said leg window during final stage of inserting said fastening protrusion into said blade socket to thereby fix said fastening protrusion in said blade socket and said blade on said blade holder.

100. The intubation laryngoscope of claim 97, wherein
said illumination means is connected with said blade holder,
said illumination means along with said blade holder is fully detachable from said blade,
the same single said illumination means provides the needed illumination of a zone of endotracheal tube insertion for any blade of said blade set, and includes
a single light extender
fastened to said blade holder,
adapted for operation with any blade of said blade set,
having a distal end emitting said illumination light and extended from said blade holder in a distal direction sufficiently to provide the needed illumination for any blade of said blade set,
disposed in said fastening protrusion of blade holder,
and said blade socket insulates said single light extender along with said fastening protrusion from patient's mouth medium.

101. The intubation laryngoscope of claim 97, wherein said blade socket includes proximally arranged lower, lateral and anterior walls completely insulating said blade holder from any contact with patient an endotracheal tube during the intubation procedure to prevent said blade holder from infecting.

102. An intubation laryngoscope comprising
a handle,
at least one detachable blade, having
a blade distal end portion designed to expose a tracheal entrance in order to insert an endotracheal tube therein,
a blade proximal end portion,
an upper longitudinal part having the form of a thin-walled sheet of various outlines and curvature,
a lateral longitudinal part disposed laterally and below relative to said upper longitudinal part,
a fastening means designed for detachable connection of said blade proximal end portion to said handle and comprising
a blade fastened section in the form of a thin-walled sheet portion located in the limits of said blade proximal end portion,
at least one blade engagement element
disposed at said blade fastened section,
a blade holder disposed at an end of said handle and including
a holder slit adapted to inserting said blade fastened section therein with an insignificant gap to partial fix said blade in said blade holder,
at least one holder engagement element, adapted to detachable engagement with said blade engagement element,
an illumination means designed to illuminate the zone of said endotracheal tube insertion.

103. The intubation laryngoscope of claim 102, wherein said blade engagement element is made as at least one engaging opening in said blade fastened section, and said holder engagement element is made as an least one movable engaging projection entering said engaging opening to attach said blade to said blade holder and coming out from said engaging opening to detach said blade from said blade holder.

104. The intubation laryngoscope of claim 102, wherein
each of said blade end portions is adapted to use as said blade distal end portion for immediate exposing said tracheal entrance,
said blade fastened section and blade engagement element are disposed on each of said blade end portions, so that each of said blade end portions is capable of applying both as said blade distal end portion designed for immediate exposing said tracheal entrance and as said blade proximal end portion designed for connecting to said handle through said blade holder, allowing previous turning said blade through 180° to convert said blade proximal end portion into said blade distal end portion and simultaneously to change the lateral disposition of said blade lateral longitudinal part relative to said upper longitudinal part in order to change the operator's hand to be used for inserting said endotracheal tube,
said blade holder is capable of detachable connecting said blade to said handle regardless of what blade end portion is chosen as said blade distal end portion,
and said illumination means is adapted to illuminate said zone of endotracheal tube insertion regardless of what blade end portion is chosen as said blade distal end portion.

105. The intubation laryngoscope of claim 102, wherein said fastening means includes lateral restrictors of a lateral movement of said blade fastened section in said holder slit made as bended continuations of said blade fastened section, which in the blade attached state are disposed beyond said holder slit adjacent to it.

106. The intubation laryngoscope of claim 102, wherein said blade engagement element and holder engagement element form a latch including movable resilient leg located on said blade and comprising said blade engagement element entering into detachable engagement with said holder engagement element immovable disposed on said blade holder.
